(19) United States
(12) Patent Application Publication
(10) Pub. No.: US 2022/0401588 A1
(43) Pub. Date: Dec. 22, 2022

(54) SIMULTANEOUS IMAGE REPRESENTATION OF TWO DIFFERENT FUNCTIONAL AREAS

(71) Applicants: Siemens Healthcare GmbH, Erlangen (DE); Bayer AG, Leverkusen (DE)

(72) Inventors: Thomas FLOHR, Uehlfeld (DE); Bernhard SCHMIDT, Fuerth (DE); Gregor JOST, Berlin (DE); Hubertus PIETSCH, Kleinmachnow (DE)

(73) Assignees: Siemens Healthcare GmbH, Erlangen (DE); Bayer AG, Leverkusen (DE)

(21) Appl. No.: 17/779,614

(22) PCT Filed: Nov. 4, 2020
(86) PCT No.: PCT/EP2020/080914

§ 371 Date: May 25, 2022

(30) Foreign Application Priority Data
Nov. 29, 2019 (DE) 10 2019 218 589.4

(51) Int. Cl. A61K 49/04; A61B 6/00

(52) U.S. Cl. CPC A61K 49/0409; A61B 6/481; A61B 6/482; A61B 6/4241; A61B 6/032

(57) ABSTRACT

An ensemble of at least two X-ray contrast agents includes X-ray contrast agent and a second X-ray contrast agent. The second X-ray contrast agent has an X-ray absorption whose change between at least two different X-ray photon energies differs significantly from the change of the X-ray absorption of the first X-ray contrast agent between the at least two different X-ray photon energies. An X-ray imaging method, an image reconstruction device, an X-ray imaging system are also disclosed.

### CROSS-REFERENCE TO RELATED APPLICATION(S)

This application is the National Phase under 35 U.S.C. § 371 of International Application No. PCT/EP2020/080914, which has an international filing date of Nov. 4, 2020, and which designated the United States of America, and which claims priority to German Application No. DE 10 2019 218 589.4, filed Nov. 29, 2019, the entire contents of each of which are incorporated herein by reference.

### FIELD

Embodiments of the present invention relate to an ensemble of at least two X-ray contrast agents. In addition, embodiments of the present invention relate to an X-ray imaging method in which said ensemble of at least two X-ray contrast agents is used. Embodiments of the present invention also relate to an image reconstruction facility. Embodiments of the present invention further relate to an X-ray imaging system.

### BACKGROUND

With the aid of modern imaging methods, two or three-dimensional image data is often created which can be used for visualizing a mapped examination object and also for other uses.

The imaging methods are often based upon the capture of X-ray radiation, wherein so-called projection scan data is generated. For example, projection scan data can be acquired with the aid of a computed tomography (CT) system.

In an X-ray image recording, contrast agents are often used which are injected into the patient in order to enhance the contrast of the image recording and thereby to facilitate a diagnosis. An example for the use of contrast agents is the representation of vessels with X-ray methods. X-ray methods can therein be carried out with conventional systems, C-arm systems, angiography systems or CT systems. Conventionally, iodine is used as an X-ray contrast agent for such an imaging process.

Uses exist wherein it would be desirable, in addition to the intravenously administered contrast agent for the representation of a local blood flow, also to use a second contrast agent for image representation in order, simultaneously, to be able to represent two different functional regions, for example, with the aid of a dual energy imaging method.

A problem of this type arises, for example, in the use of a chemoembolization to treat liver tumors. In a treatment of this type, shortly after the chemoembolization, representation of the remaining local blood flow in the tumor, also known as perfusion, takes place. The extent of the local blood flow through the tumor therein represents a measure of the success of this method. This means that the lower the blood flow in the tumor region, the more effective is the treatment. In chemoembolization, an administration of a chemotherapy agent is combined with a simultaneous targeted blocking of arteries in the liver via small particles such as oily droplets. For the representation of the embolization region, that is, the blocked region, the material for embolization, for example Lipiodol, itself comprises a contrast agent. The contrast agent remains with the embolic agent in the region of the liver. If, in addition to the representation of the local blood flow after the embolization, a second contrast agent is administered, then the regions affected by the two contrast agents must be able to be represented separately from one another. If the iodine-based Lipiodol is used for embolization and iodine is used as the second contrast agent, then a separation is not possible or only with difficulty, since the contrast agents mentioned behave similarly with regard to their absorption and/or their absorption spectrum.

A possibility for nevertheless separating the two contrast agents lies in using so-called subtraction techniques in which a CT prescan of a patient is created after the chemoembolization but before the administration of the intravenous contrast agent, and is subtracted from a CT scan that takes place after an administration of the intravenous contrast agent so that in the image representation only the intravenously administered contrast agent remains visible. However, such a subtraction requires a precise registration of the image data of the two scans to compensate for patient movement. Furthermore, due to the increased number of CT image recordings, the radiation dose to the patient is increased.

Alternatively, as a second contrast agent, a contrast agent can be used that is not based on iodine. However, conventionally, only gadolinium has been available in addition. Gadolinium has a K-edge at approximately 50 keV and has a spectral behavior very similar to that of iodine the K-edge of which is at 33 keV, so that a two-material decomposition on the basis of a dual-energy imaging process results in an iodine image and a gadolinium image in a very imprecise manner, and is associated with very intense noise and a very poor material separation, so that it is unusable in clinical practice.

An improved separation of the image regions imaged by the two contrast agents iodine and gadolinium can be achieved, for example, by way of CT scans with more than two energies, for example by the use of photon-counting detectors. In imaging of this type, with the selection of three energies, a decomposition into an iodine image, a gadolinium image and a soft tissue image can take place. However, in the case of a decomposition according to three materials, a high level of image noise also occurs, which can only be compensated for by increasing the radiation dose for the patient. Furthermore, a recording with at least three energies is possible only with CT systems having photon-counting detectors which, however, are not available very often.

The simultaneous application of two contrast agents is also used for the simultaneous representation of the arterial phase and the venous or portal venous phase of a liver CT examination in separate images which are calculated on the basis of CT data from a single CT scan. For the simultaneous image recording thereof, two different contrast agents are injected offset temporally before the CT scan. Therein, a first contrast agent is injected sufficiently early that at the time of the CT scan, it has already reached the venous and/or portal venous phase and a second contrast agent is injected correspondingly later so that at the time point of the CT scan, it maps the arterial phase. In this application, also, it is necessary to be able to distinguish the two contrast agents clearly from one another in the image recording. However, the two conventionally available contrast agents iodine and gadolinium are so similar in their spectral absorption behavior that they cannot be separated well from one another with dual-energy image recordings. Although three-material decompositions are possible with the aid of CT systems having photon-counting detectors, the problems of increased noise and the need for an increase in the radiation dose to the patient also arise.

A simultaneous representation with two contrast agents is necessary also in the determination of the lung perfusion with simultaneous representation of the lung ventilation. Therein, imaging of the local blood flow in the lung parenchyma as a measure of the lung perfusion takes place by intravenous administration of a first contrast agent and simultaneously therewith, the lung ventilation is made visible by inhalation of a second contrast agent. Normally, iodine is used as the contrast agent for representing the local blood flow in the lung parenchyma and as the contrast agent for representing lung ventilation, xenon is used. However, xenon behaves very similarly to iodine with regard to its spectral absorption behavior so that a dual-energy CT image recording and/or a two-material separation into an iodine image and a xenon image based thereupon provides no usable results.

### SUMMARY

Conventionally, a separate representation of two different contrast agents in CT images can be realized only by subtraction techniques or spectral CT scans with at least three energies, which can only be carried out with photon-counting detectors. However, the methods mentioned are associated with a higher radiation dose to the patient in comparison with dual-energy CT imaging and the image representation, which is based upon a three-material decomposition, is also heavily laden with noise and is therefore only seldom purposefully usable.

There therefore exists the problem of realizing a qualitatively good simultaneous image representation of functional regions with a plurality of contrast agents and an acceptable radiation dose.

Embodiments of the present invention achieve this with an ensemble of X-ray contrast agents, an X-ray imaging method, an image reconstruction facility and an X-ray imaging system.

The ensemble of X-ray contrast agents according to embodiments of the present invention has a first X-ray contrast agent and a second X-ray contrast agent. The second X-ray contrast agent has an X-ray absorption the change of which between at least two different X-ray photon energies differs significantly from the change in the X-ray absorption of the first contrast agent between the at least two different X-ray photon energies. It can be stated in this regard that the absorption of X-ray contrast agents can change dependent upon the energy of the X-ray photons. However, conventional contrast agents such as iodine and gadolinium have a very similar change behavior so that they are not able to be represented effectively separated from one another. According to embodiments of the present invention, two X-ray contrast agents which have a different behavior of change in their absorption dependent upon the energy of the incident X-ray photons and are therefore distinguishable from one another in a multi-energy CT image recording, in particular a dual-energy CT image recording, are to be combined with one another for simultaneous image representation.

“Significantly” should be understood in this context to mean that the change in the absorption of the second X-ray contrast agent amounts to less than half the change in the first X-ray contrast agent at the selected different X-ray photon energies.

Advantageously, the spectrally deviating behavior of the second contrast agent according to embodiments of the present invention can be used to represent regions which are flooded by the second contrast agent separately from other image regions that are affected by the first contrast agent. This means that it is achieved that the two contrast agents can be clearly distinguished from one another in a joint image recording. Thereby, the accuracy of a simultaneous representation of two different functional regions and/or two different functional processes in an examination region is improved as compared with conventionally used contrast agents.

In the X-ray imaging method according to embodiments of the present invention, initially a selection of an ensemble of at least two X-ray contrast agents according to embodiments of the present invention takes place. Furthermore, X-ray raw data is captured from a region of an examination object which is flooded by a first X-ray contrast agent and from a region of the examination object which is flooded by a second X-ray contrast agent, with the aid of a multi-energy recording method, preferably a dual-energy recording method. As mentioned above, a dual-energy CT image recording is associated with a lower noise effect than CT image recordings with a larger number of different energies and/or a greater number of simultaneous recordings with different X-ray spectra. Then, a material decomposition takes place on the basis of the X-ray raw data relating to the two X-ray contrast agents. The X-ray imaging method according to embodiments of the present invention can be carried out as a computer-implemented method on the basis of the captured data.

Material decomposition, which is known in principle, proceeds from the consideration that an X-ray attenuation value measured by an X-ray image recording apparatus can be described as a linear combination of X-ray attenuation values of so-called base materials with regard to the aforementioned X-ray quantum energy distribution and/or X-ray photon energy. Measured X-ray attenuation values result from the at least two raw datasets and/or image datasets reconstructed therefrom at different X-ray quantum energy distributions. The material and/or base material in the application according to embodiments of the present invention are the two X-ray contrast agents. The X-ray attenuation of a base material dependent upon the energy of the X-ray radiation is, in principle, known or can be determined by way of prior measurements with phantoms and stored in the form of tables for retrieval in the context of the material decomposition. The result of the material decomposition is a spatial density distribution of the at least two materials, i.e. of the X-ray contrast agent according to embodiments of the present invention, from which for each volume element in the body region of the patient that is to be mapped, the base material proportions and/or the base material combination can be ascertained.

The material decomposition can both relate directly to the raw data and can also take place on the basis of the reconstructed image data. In any event, in the context of the method, at least two image datasets are generated on the basis of spectrally decomposed data, whether raw data or image data: the at least two image datasets comprise a first image dataset, which represents a first image region which is affected by the first contrast agent, and a second image dataset, which preferably represents a second image region which is complementary to the first image region and which is affected by the second X-ray contrast agent.

In any event, at least two image datasets are reconstructed on the basis of the material decomposition. The two image datasets comprise a first image dataset, which represents a first image region which is affected by the first contrast agent, and a second image dataset, which represents a second image region which is affected by the second contrast agent.

In the case of a complementary representation of the first and the second image dataset, regions affected by the first and the second X-ray contrast agent can be visualized together in one image, for example, by way of an overlaying of the two image datasets, wherein the relative position of the different functional regions and the spatial separation and/or boundary surfaces between these different regions are readily recognizable.

If the different materials represented by the two image datasets and/or the X-ray contrast agents making them visible are present intermingled, then for separate visualization of the different X-ray contrast agents and/or the structures and/or physical functions made visible thereby, a separate representation of each of the first and the second image datasets in two separate images can also take place.

The X-ray imaging method according to embodiments of the present invention enables a precise simultaneous representation with two simultaneously utilized contrast agents.

The image reconstruction facility according to embodiments of the present invention has an ascertaining unit for ascertaining at least two different X-ray photon energies. The at least two different X-ray photon energies are selected so that, at these energies, a first contrast agent differs significantly from a second contrast agent with regard to the change in the X-ray absorption between the at least two different X-ray photon energies.

The selection of the energy values can take place, for example, on the basis of stored energy-dependent absorption values of the selected contrast agents. The selection of the energy values can be taken into account, in the context of a multi-energy recording method, in the selection of the energies and/or the mean energy values of the X-ray sources used for imaging. If counting detectors are used for capturing the X-ray radiation, then energy thresholds and/or intervals can be selected so that the energy values mentioned are included.

A part of the image reconstruction facility according to embodiments of the present invention is a raw data receiving unit for receiving X-ray raw data from a region of an examination object which is flooded by the first contrast agent and from a region of the examination object which is flooded by the second contrast agent, with the aid of a multi-energy recording method, preferably a dual-energy imaging method.

The image reconstruction facility according to embodiments of the present invention also comprises a decomposition unit for carrying out a material decomposition on the basis of the X-ray raw data relating to the two X-ray contrast agents. Furthermore, the image reconstruction facility according to embodiments of the present invention also comprises a reconstruction unit for reconstructing at least two image datasets on the basis of the material decomposition. The image datasets comprise a first image dataset, which represents a first image region which is affected by the first contrast agent, and a second image dataset, which represents a second image region which is affected by the second X-ray contrast agent. The image reconstruction facility according to embodiments of the present invention shares the advantages of the X-ray imaging method according to embodiments of the present invention.

The X-ray imaging system according to embodiments of the present invention has an image reconstruction facility according to embodiments of the present invention. The X-ray imaging system according to embodiments of the present invention can preferably comprise a CT system.

The essential components of the image reconstruction facility according to embodiments of the present invention can be configured mainly in the form of software components. This relates, in particular, to the decomposition unit and the reconstruction unit of the image reconstruction facility according to embodiments of the present invention. Fundamentally however, these components can also, in part, be realized in particular if particularly rapid calculations are involved, in the form of software-supported hardware, for example, FPGAs or the like. Similarly, the required interfaces can be configured, for example, where only an acceptance of data from other software components is concerned, as software interfaces. However, they can also be configured as interfaces which are constructed as hardware and are controlled by suitable software.

A realization largely with software has the advantage that medical technology X-ray imaging systems and/or image reconstruction facilities which are already conventionally used can also be upgraded easily with a software update in order to operate in the manner according to embodiments of the present invention. In this respect, the object is also achieved by a corresponding computer program product with a computer program which can be loaded directly into a storage facility of an X-ray imaging system, having program portions in order to carry out the steps of the X-ray imaging method according to embodiments of the present invention that can be realized with software when the program is executed in the X-ray imaging system. Such a computer program product can comprise, apart from the computer program, additional components, if relevant, such as for example documentation and/or additional components including hardware components, for example hardware keys (dongles, etc.), in order to use the software.

For transport to the X-ray imaging system and/or for storage at or in this X-ray imaging system, a computer-readable medium, for example a memory stick, a hard disk or another transportable or permanently installed data carrier can be used on which the program portions of the computer program which can be read in and executed by a computer unit are stored. For this purpose, the computer unit can have, for example, one or more cooperating microprocessors or the like. The computer unit can be, for example, part of a terminal or a control facility of an X-ray imaging system, for example a CT system, but can also be part of a remotely arranged server system within a data transfer network which communicates with the X-ray imaging system.

The dependent claims and the description below each contain particularly advantageous embodiments and developments of embodiments of the present invention. In particular, the claims of one claim category can also be developed similarly to the dependent claims of another claim category. In addition, in the context of the disclosure, the different features of different example embodiments and claims can also be combined to form new example embodiments.

In a variant of the ensemble of X-ray contrast agents according to embodiments of the present invention, the X-ray absorption of the first contrast agent for the at least two X-ray photon energies is significantly different and the X-ray absorption of the second contrast agent for the at least two X-ray photon energies is not significantly different.

“Not significantly different” should be understood in this context to mean that the change in the absorption of the second X-ray contrast agent amounts to less than half the change in the absorption of the first X-ray contrast agent at the selected different X-ray photon energies.

Advantageously, the two X-ray contrast agents according to embodiments of the present invention differ from one another with regard to their absorption behavior dependent upon the photon energy. As described above, this different absorption behavior can be used to differentiate the two X-ray contrast agents from one another in the imaging.

Particularly preferably, the X-ray absorption of the second X-ray contrast agent is similar to the spectrum of the X-ray absorption of water or soft tissue. Naturally, the second contrast agent should have a greater absorption than is the case for water or soft tissue. In this regard, the similarity should therefore not relate to absolute values of absorption, but to the change in the absorption dependent upon the X-ray photon energy. The reason is that, in an energy range that is relevant for CT imaging, water or soft tissue exhibit a behavior which is independent of the photon energy and can therefore easily be separated from conventional contrast agents, such as for example iodine or gadolinium.

In a particularly preferred embodiment of the ensemble of at least two X-ray contrast agents according to embodiments of the present invention, the first contrast agent has one of the following materials:
iodine,
gadolinium
   and the second contrast agent has one of the following materials:
tungsten,
tantalum,
hafnium,
gold.

The materials selected for the second contrast agent all advantageously have a water-like absorption behavior. For this reason, subregions of an examination region affected and/or flooded by the second contrast agent can easily be separated or represented separately from iodine-containing or gadolinium-containing regions.

In one embodiment of the X-ray imaging method according to embodiments of the present invention, it has a multi-energy imaging method, preferably a dual-energy imaging method, in which at least two different X-ray tube voltages, at which the change in the absorption of the first and the second contrast agent differs significantly, are specified.

Furthermore, at least two datasets of X-ray image recordings are carried out with the at least two different X-ray tube voltages for acquisition of a first raw dataset and at least one second raw dataset. The material decomposition then takes place on the basis of the at least two raw datasets. In this variant, with the aid of different X-ray tube voltages, X-rays with different X-ray spectra are generated. These are used for generating at least two raw datasets which are used for separating different contrast agents during the imaging.

In this embodiment, at least two X-ray image recordings are carried out with the at least two different X-ray tube voltages.

In an alternative embodiment of the X-ray imaging method according to embodiments of the present invention, X-ray raw data which has been recorded with the aid of a photon-counting detector in an energy-resolved manner is captured, wherein the energy thresholds of the photon-counting detector are set such that therein, the change in the absorption of the first contrast agent differs significantly from the change in the absorption of the second contrast agent. Furthermore, a material decomposition takes place on the basis of the energy-resolved raw data. Advantageously, in this variant, only the irradiation of an examination region with just one single X-ray tube is needed since the spectral separation of the X-ray radiation takes place in the detector.

Preferably, the X-ray imaging method according to embodiments of the present invention comprises one of the following CT imaging methods:
a simultaneous representation of an embolic agent and a local blood flow during a chemoembolization,
a simultaneous representation of a venous or portal venous phase and an arterial phase of a liver,
a simultaneous representation of a local blood flow of a lung parenchyma and a lung ventilation.

Advantageously, the examinations mentioned can be realized with the X-ray imaging method according to embodiments of the present invention with a lower radiation dose and improved image quality as compared with the conventional procedure.

### BRIEF DESCRIPTION OF THE DRAWINGS

The present invention will now be described again in greater detail using example embodiments, making reference to the accompanying figures, in which:

FIG. 1 shows a graphical representation that illustrates absorption values of the contrast agent iodine and the material tungsten dependent upon the tube voltage of an X-ray facility,

FIG. 2 shows a graphical representation that represents the absorption properties of the contrast agents iodine and tungsten and of calcium and water dependent upon the energy of the X-ray photons,

FIG. 3 shows a flow diagram that illustrates an X-ray imaging method according to a first example embodiment of the present invention,

FIG. 4 shows a schematic representation of an image reconstruction facility according to an example embodiment of the present invention,

FIG. 5 shows a schematic representation of a CT system according to an example embodiment of the present invention.

### DETAILED DESCRIPTION

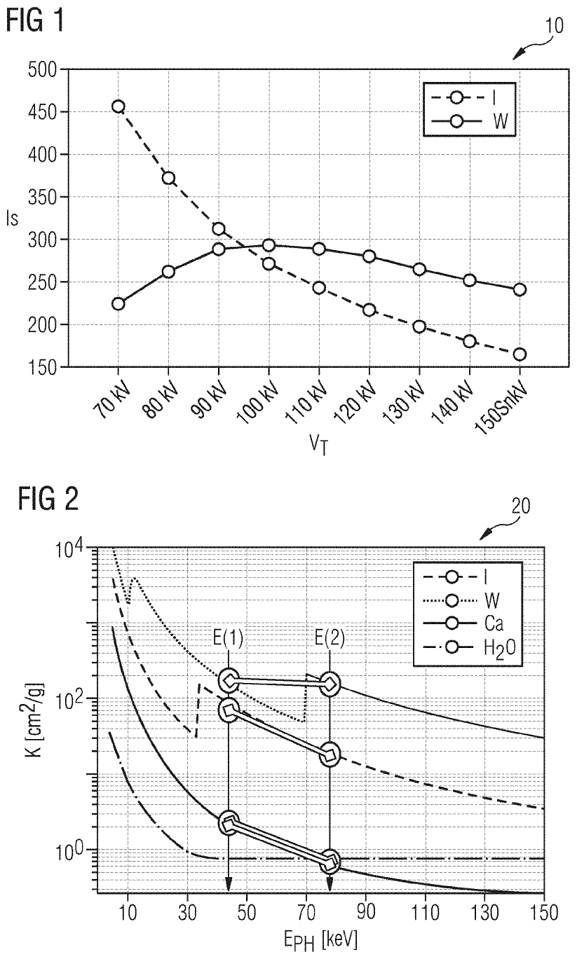

FIG. 1 shows a graphical representation 10 that illustrates absorption values Is of the contrast agent iodine I and the material tungsten W dependent upon the tube voltage VT of an X-ray facility. Whereas the X-ray absorption of iodine I decreases with increasing energy, the X-ray absorption of tungsten W decreases only slightly with increasing energy. Particularly in a dual-energy image recording at a low energy of 80 kV and a higher energy of 140 kV or 150 kV with a tin filter, the X-ray absorption Is of tungsten W changes practically not at all as compared with the X-ray absorption of iodine I. Therefore, image points at which the two individual recordings are generated with different tube voltages can easily be associated with one of the two contrast agents. For example, a point at which the absorption is the same in the two images is clearly attributable to the material tungsten W and a point at which the absorption in the two images is strongly different is clearly attributable to the material iodine I.

FIG. 2 shows a graphical representation 20 that illustrates absorption properties of the contrast agents iodine I and tungsten W as well as those of calcium Ca and water H2O, dependent upon the energy EPH of the X-ray photons. For each of the materials mentioned, the mass absorption coefficient K is shown dependent upon the energy EPH of the X-ray photons. It is clearly apparent in FIG. 2 that the absorption of the contrast agent iodine I and of the bone material calcium Ca decreases strongly in the region from 40 to 80 keV with increasing photon energy EPH. It should be noted that therein the absorption is shown logarithmically. In contrast thereto, tungsten W behaves more like water H2O. That is, the absorption for a first photon energy E(1), which is at approximately 45 keV, is equal to the absorption at a second photon energy E(2) which is at approximately 80 keV. Due to the strongly differing behavior of tungsten W as compared with calcium Ca, image regions which are laden with tungsten W can readily be separated or separately represented from regions in which calcium Ca prevails.

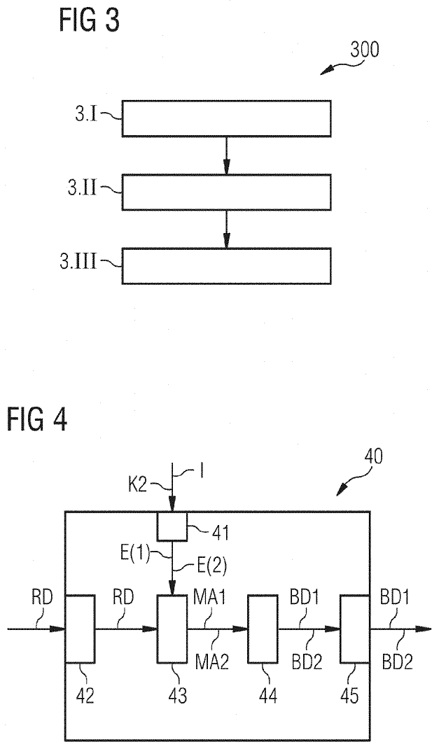

FIG. 3 shows a flow diagram 300 which illustrates an X-ray imaging method according to an example embodiment of the present invention. In the example embodiment according to FIG. 3, an imaging of a chemoembolization of a tumor in the liver is to be carried out.

For this purpose, an ensemble of two contrast agents is selected in step 3.I, specifically the iodine-based Lipiodol and an intravenous contrast agent based upon the element tungsten.

Furthermore, in the step 3.II, X-ray raw data RD is captured from a region affected by Lipiodol, i.e. the edge region of the tumor and a region flooded with the intravenous contrast agent, with the aid of a dual-energy recording method. In the method visualized in FIG. 3, X-ray raw data that has been recorded with X-ray radiation at two different energy values E(1) and E(2) is captured. The energy values are therein selected such that the absorption behavior of the intravenous contrast agent, based in this example embodiment upon the material tungsten, is the same for both the energy values.

The image recording process can be realized, for example, by way of the use of two detectors arranged spatially separated from one another, wherein a filter is introduced into the beam path in front of one of the two detectors, said filter filtering out part of the spectrum of the X-rays. Therefore, two raw datasets are captured with different X-ray photon spectra.

In step 3.III, a reconstruction of two image datasets BD1, BD2 takes place on the basis of the two raw datasets.

The reconstruction takes place on the basis of a material decomposition according to the two contrast agents used.

A first image dataset BD1 represents a first image region affected by the Lipiodol and a second image dataset BD2 represents a second image region affected by the tungsten-based contrast agent. The two image regions are easily distinguishable from one another in a common image representation due to the strongly different properties of the contrast agents used.

FIG. 4 shows a reconstruction facility 40. The reconstruction facility 40 has an establishing unit 41. The establishing unit 41 receives information regarding the contrast agents I, K2 to be used and establishes values E(1), E(2) of two different X-ray photon energies at which a selected contrast agent K2 behaves like water, i.e. the absorption is the same for both energy values. However, the image regions affected by iodine I that are to be separated from the contrast agent K2 have a dependence of the absorption on the X-ray photon energy and, due to the different absorption behavior, can therefore easily be differentiated at the established energy values E(1), E(2) from the selected contrast agent K2. The selection of the energy values can take place, for example, on the basis of stored energy-dependent absorption values of the selected contrast agent K2. The selection of the energy values E(1), E(2) can be taken into account, in the context of a multi-energy recording method, in the selection of the energy of the X-ray sources used for imaging. If counting detectors are used for capturing the X-ray radiation, then energy thresholds and/or intervals can be selected so that the energy values mentioned are included.

The reconstruction facility 40 also has a raw data receiving unit 42 for receiving X-ray raw data RD. The raw data RD has been acquired with the aid of a dual-energy CT method from a region of an examination object which is at least partially flooded by the contrast agents I, K2.

The raw data RD is passed on to a decomposition unit 43 which carries out a material decomposition on the basis of the X-ray raw data RD in relation to the contrast agents I, K2. The portions MA1, MA2 which are associated with the individual absorption spectra of the different materials are transferred to a reconstruction unit 44 which reconstructs at least two image datasets BD1, BD2 on the basis of the material-specific portions MA1, MA2. A first image dataset BD1 visualizes a first image region affected by the tungsten-based contrast agent K2 and a second image dataset BD2 visualizes a second image region which is complementary to the first image region, and in which structures contrasted with iodine prevail. The image data BD1, BD2 are finally output via an output interface 45.

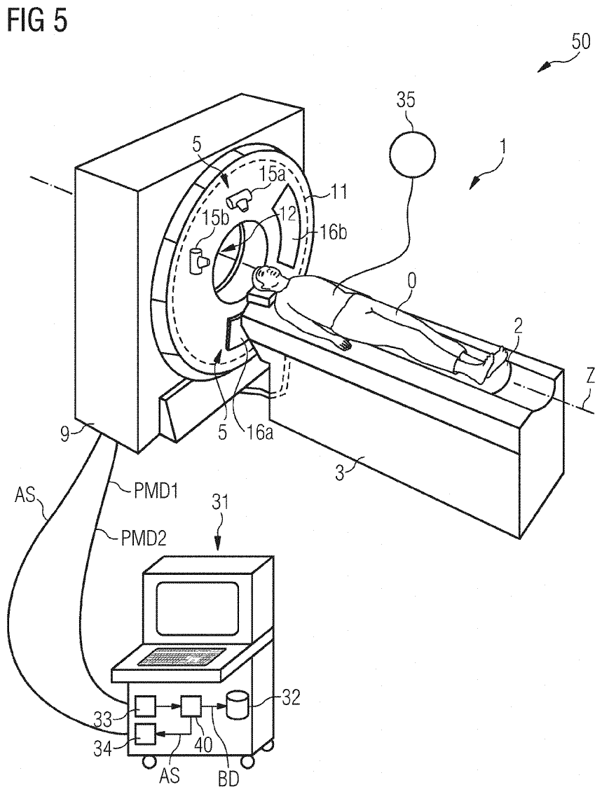

FIG. 5 visualizes an X-ray imaging system, in this case a CT system 50, according to an example embodiment of the present invention.

The CT system 50 which is configured as a dual-energy CT system, substantially consists therein of a typical scanner 9 in which a projection measurement data acquisition unit 5 with two detectors 16a, 16b and two X-ray sources 15a, 15b arranged opposite the two detectors 16a, 16b circulates on a gantry 11 around a scanning space 12. Situated in front of the scanner 9 is a patient positioning apparatus 3 and/or a patient table 3, the upper part 2 of which can be displaced with a patient o situated thereon toward the scanner 9, in order to move the patient o through the scanning space 12 relative to the detector system 16a, 16b. The scanner 9 and the patient table 3 are controlled by way of a control facility 31 from which acquisition control signals AS come via a conventional control interface 34 in order to control the whole system according to predetermined scan protocols in the conventional manner. In the case of a spiral acquisition, by way of a movement of the patient o along the z-direction which corresponds to the system axis z through the scanning space 12 and the simultaneous circulation of the X-ray sources 15a, 15b, for the X-ray sources 15a, 15b relative to the patient o during the scan, a helical path results. The detectors 16a, 16b therein always move in parallel opposite to and with the X-ray sources 15a, 15b, in order to capture projection measurement data PMD1, PMD2 which is then used for the reconstruction of volume and/or slice image data. Similarly, a sequential scanning method can also be carried out in which a fixed position in the z-direction is moved to and then, during a circulation, a partial circulation or a plurality of circulations at the z-position in question, the required projection measurement data PMD1, PMD2 is captured, in order to reconstruct a slice image at this z-position or to reconstruct image data from the projection measurement data of a plurality of z-positions. The method according to embodiments of the present invention is also in principle usable with other CT systems, for example, with just one X-ray source or with a detector forming a complete ring. For example, the inventive method can also be used on a system with an unmoved patient table and a gantry moved in the z-direction (a so-called sliding gantry).

The projection measurement data PMD1, PMD2 (also referred to here as raw data) acquired from the detectors 16a, 16b is transferred via a raw data interface 33 to the control facility 31. This raw data is then further processed, possibly after a suitable pre-processing in a reconstruction facility 40 which, in this example embodiment, is realized in the control facility 31 in the form of software on a processor. This reconstruction facility 40 reconstructs, on the basis of the raw data PMD1, PMD2, two image datasets BD1, BD2, of which a first image dataset BD1 represents structures affected by a first X-ray contrast agent according to embodiments of the present invention, for example a tungsten-based contrast agent, and a second image dataset BD2 represents image regions affected by a second contrast agent according to embodiments of the present invention, for example iodine.

The precise construction of such a reconstruction facility 40 is illustrated in detail in FIG. 4.

The image data BD1, BD2 generated by the reconstruction facility 40 is then stored in a memory store 32 of the control facility 31 and/or is output in the usual manner on the screen of the control facility 31. Via an interface (not shown in FIG. 5), it can also be fed into a network connected to the computed tomography system 50, for example, a radiological information system (RIS), and stored in a mass memory store accessible there or output as images to printers or filming stations connected there. The data can thus be further processed in any desired manner and then stored or output.

In addition in FIG. 5, a contrast agent injection facility 35 is shown, with which the two contrast agents according to embodiments of the present invention are injected into the patient o in advance, that is, before the start of the CT imaging process. The regions which are flooded by the contrast agents can then be captured in image form with the aid of the computed tomography system 50 using the X-ray imaging method according to embodiments of the present invention.

The components of the reconstruction facility 40 can be realized mainly or entirely in the form of software elements on a suitable processor. In particular, the interfaces between these components can also be configured purely as software. It is required only that access possibilities exist in suitable memory storage regions in which the data can suitably be placed in intermediate storage and, at any time, called up again and updated.

Finally, it should again be noted that the methods and apparatuses described above are merely preferred example embodiments of the present invention and that the present invention can be modified by a person skilled in the art without departing from the field of embodiments of the present invention, to the extent that it is specified by the claims. For the sake of completeness, it should also be noted that the use of the indefinite article “a” or “an” does not preclude the relevant features from being present plurally. Similarly, the expression “unit” does not preclude this consisting of a plurality of components which can possibly also be spatially distributed.

1. An X-ray imaging system including an ensemble of at least two X-ray contrast agents, the ensemble comprising:
a first X-ray contrast agent having a first X-ray absorption; and
a second X-ray contrast agent having a second X-ray absorption, a change of the second X-ray absorption between at least two different X-ray photon energies differing significantly from a change in the first X-ray absorption between the at least two different X-ray photon energies.

2. The X-ray imaging system of claim 1, wherein
the first X-ray absorption of the first X-ray contrast agent for the at least two different X-ray photon energies is significantly different, and
the second X-ray absorption of the second X-ray contrast agent for the at least two different X-ray photon energies is not significantly different.

3. The X-ray imaging system of claim 2, wherein a spectrum of the second X-ray absorption of the second X-ray contrast agent is similar to a spectrum of an X-ray absorption of water or soft tissue.

4. The X-ray imaging system of claim 1, wherein
the first X-ray contrast agent includes
iodine, or
gadolinium; and
the second X-ray contrast agent includes
tungsten,
tantalum,
hafnium, or
gold.

5. An X-ray imaging method, comprising:
selecting an ensemble of X-ray contrast agents, the ensemble including a first X-ray contrast agent having a first X-ray absorption, and a second X-ray contrast agent having a second X-ray absorption a change of the second X-ray absorption between at least two different X-ray photon energies differing significantly from a change in the first X-ray absorption between the at least two different X-ray photon energies,
capturing, with the aid of a multi-energy recording method, X-ray raw data from a region of an examination object which is flooded by the first X-ray contrast agent and from a region of the examination object which is flooded by the second X-ray contrast agent,
carrying out a material decomposition based on the X-ray raw data in relation to the first X-ray contrast agent and the second X-ray contrast agent, and
reconstructing at least two image datasets based on the material decomposition, the at least two image datasets including
a first image dataset representing first image region affected by the first X-ray contrast agent, and;
a second image dataset representing a second image region affected by the second X-ray contrast agent.

6. The X-ray imaging method as claimed in claim 5, wherein the multi-energy recording method comprises:
specifying at least two different X-ray tube voltages at which a change in the first X-ray absorption of the first X-ray contrast agent and the second X-ray absorption of the second X-ray contrast agent differs significantly,
capturing at least two datasets of X-ray image recordings with the at least two different X-ray tube voltages for acquisition of a first raw dataset and at least one second raw dataset, and
carrying out the material decomposition based on the first raw dataset and the at least one second raw dataset.

7. The X-ray imaging method as claimed in claim 5, wherein
the capturing of the X-ray raw data takes place by way of an energy-resolved capture of X-ray raw data with the aid of a photon-counting detector, energy thresholds of the photon-counting detector being set such that the change in the first X-ray absorption of the first X-ray contrast agent differs significantly from the change in the second X-ray absorption of the second X-ray contrast agent, and
the material decomposition is based on energy-resolved raw data.

8. The X-ray imaging method as claimed in claim 5, wherein the X-ray imaging method is one of the following CT imaging methods
a simultaneous representation of an embolic agent and a local blood flow during a chemoembolization,
a simultaneous representation of a venous or portal venous phase and an arterial phase of a liver, or
a simultaneous representation of a local blood flow of a lung parenchyma and a lung ventilation.

9. An image reconstruction facility, comprising:
an establishing unit to ascertain at least two different X-ray photon energies at which a first X-ray contrast agent differs significantly from a second X-ray contrast agent with regard to a change in an X-ray absorption between the at least two different X-ray photon energies,
a raw data receiving unit to receive X-ray raw data from a region of an examination object which is flooded by the first X-ray contrast agent and from a region of the examination object which is flooded by the second X-ray contrast agent, with the aid of a multi-energy recording method,
a decomposition unit to carry out a material decomposition based on the X-ray raw data in relation to the first X-ray contrast agent and the second X-ray contrast agent,
a reconstruction unit to recontruct at least two image datasets based on the material decomposition, the at least two image datasets including
a first image dataset to represeting a first image region affected by the first X-ray contrast agent, and
a second image dataset representing a second image region affected by the second X-ray contrast agent.

10. An X-ray imaging system, having an image reconstruction facility as claimed in claim 9.

11. The X-ray imaging system as claimed in claim 10, having a CT imaging facility.

12. A non-transitory program product including a computer program directly loadable into a storage facility of an X-ray imaging system, the non-transitory computer program product having program portions configured to cause the X-ray imaging system to carry out the method of claim 5 when the computer program is executed in the X-ray imaging system.

13. A non-transitory computer-readable medium storing program portions that, when executed by a computer unit, cause the computer unit to carry out the method as claimed in claim 5.

14. The X-ray imaging method of claim 5, wherein the multi-energy recording method is a dual-energy recording method.

15. The X-ray imaging method of claim 6, wherein the multi-energy recording method is a dual-energy recording method.

16. The X-ray imaging system of claim 2, wherein
the first X-ray contrast agent includes
iodine, or
gadolinium; and
the second X-ray contrast agent includes
tungsten,
tantalum,
hafnium, or
gold.

17. The X-ray imaging system of claim 3, wherein
the first X-ray contrast agent includes
iodine, or
gadolinium; and
the second X-ray contrast agent includes
tungsten,
tantalum,
hafnium, or
gold.

18. The X-ray imaging method as claimed in claim 6, wherein the X-ray imaging method is one of the following CT imaging methods
a simultaneous representation of an embolic agent and a local blood flow during a chemoembolization,
a simultaneous representation of a venous or portal venous phase and an arterial phase of a liver, or
a simultaneous representation of a local blood flow of a lung parenchyma and a lung ventilation.

19. The X-ray imaging method as claimed in claim 7, wherein the X-ray imaging method is one of the following CT imaging methods
a simultaneous representation of an embolic agent and a local blood flow during a chemoembolization,
a simultaneous representation of a venous or portal venous phase and an arterial phase of a liver, or
a simultaneous representation of a local blood flow of a lung parenchyma and a lung ventilation.

20. The image reconstruction facility of claim 9, wherein the multi-energy recording method is a dual-energy recording method.
